(19) United States
(12) Patent Application Publication
(10) Pub. No.: US 2023/0355426 A1
(43) Pub. Date: Nov. 9, 2023

(54) MANDIBULAR POSITIONING DEVICE FOR CONTINUOUS POSITIVE AIRWAY PRESSURE THERAPY EQUIPMENT

(71) Applicant: Arnold Rugama, Fresno, CA (US)

(72) Inventor: Arnold Rugama, Fresno, CA (US)

(21) Appl. No.: 18/311,680

(22) Filed: May 3, 2023

Related U.S. Application Data
(60) Provisional application No. 63/338,303, filed on May 4, 2022.

(51) Int. Cl. A61F 5/56; A61M 16/06

(52) U.S. Cl. CPC A61F 5/56; A61M 16/0694

(57) ABSTRACT

A mandibular positioning device for use with headgear for Continuous Positive Airway Pressure (“CPAP”) therapy face masks. The device can be integrated with existing headgear to maintain the position of a user's mandible during sleep while using CPAP therapy in order to prevent displacement of the user's face mask as a result of a posterior shift of the mandible. The device can include a pair of saddles for attaching the device to headgear, as well as a support strap which attaches to each saddle and which has a medial portion disposed underneath the user's chin when worn.

### CROSS-REFERENCE TO RELATED APPLICATIONS

The present application claims priority to, and the benefit of, U.S. Provisional Patent Application No. 63/338,303, filed May 4, 2022, which is hereby incorporated by reference in its entirety.

### FIELD OF THE INVENTION

The present invention concerns a device for use with Continuous Positive Airway Pressure (“CPAP”) therapy equipment. More particularly, embodiments of the present invention concern a device which can be integrated with a face mask and used during CPAP therapy to maintain and stabilize the mandibular position of a user.

### BACKGROUND OF THE INVENTION

Obstructive Sleep Apnea (“OSA”) is a well-known cause of excessive daytime sleepiness and other diseases which lead to significant morbidity and mortality, including hypertension and depression. It is during the deep stages of sleep that total body muscle relaxation occurs, including posterior mandibular relaxation. This posterior relaxation of the mandible is often associated with relaxation of the tongue into the airway, which can cause an obstruction of airflow and reduced oxygen to the brain and body. This airway obstruction stimulates a cascade of physiologic responses that result in the brain transitioning to a lighter stage of sleep and, therefore, re-establishing muscle tone to obviate occlusion of the airway by the tongue. The brain's drive to return to the deep stage of sleep is paramount to good quality sleep and is the underlying reason of the constant, and often frequent, sleep stage shifts in OSA. Greater awareness of the relationship between OSA and chronic disease has accelerated diagnostic and therapeutic technologies.

The treatment of OSA has become more effective with the rapid improvements to Continuous Positive Airway Pressure (“CPAP”) machines and face mask technologies. CPAP therapy provides an “air splint” that channels positive airflow through a plastic tube which is connected to a face mask interface. The delivery of the constant air pressure prevents occlusion of the airway by the tongue. The mask is held in place on the head with headgear. Mask headgear conventionally consists of a set of upper straps that surround the head at the temple, a set of lower straps that surround the head below the ears, and a top strap over the top of the head and interconnecting the upper straps, each of which are conventionally made from narrow strips of non-woven fabric. A patient has the ability to apply tension to the straps to adjust the face mask interface into position over the nose and mouth for a stable, comfortable fit of the mask on the face.

The nightly use of this mode of therapy for the treatment of OSA typically improves sleep quality and overall health. However, there are many potential complications that could disrupt success with the nightly use of CPAP machines. Adherence to CPAP machines has been a focus of active investigation throughout the world. Although there are many components of this treatment modality, one aspect that has been given little attention to are the issues related to mandibular relaxation and its impact on optimal CPAP therapy.

When a patient uses a mask-headgear interface that covers the mouth and extends either around the nose (i.e., a full-face mask) or under the nose, air pressure enters both the nasal or oral passageways. It is during sleep that the mandible relaxes posteriorly and thus a leak of the constant air pressure around the periphery of the mask can occur. This leak decreases the required pressure to maintain the “air splint”. Air leaks are disruptive to sleep, often causing a full awakening. An air leak may cause a noisy vibration along the periphery of the mask and is often felt on the skin of the face. This disruption often results in the patient excessively tightening the mask onto the face to a point that becomes uncomfortable. The patient may then request an alternative mask interface, or may completely discontinue the use of CPAP therapy all together.

Conventional headgear applies a force to the front and back of the head, but, however, lacks any support for the chin. Therefore, one conventional approach to addressing mask leaks is the use of a chin strap. A conventional chin strap is a separate piece of equipment designed to be used with face masks which completely encircles the head from the vertex to the chin. However, conventional chin straps have achieved varied success due to issues with discomfort and/or inconvenience, as well as their design. For instance, while conventional chin straps provide an upward force on the chin to help keep the mandible in place, they are prone to sliding off at the chin or at the top of the head, as there are no forces toward the front or the back of the head to keep the chin strap in place. Other conventional chin straps may have arms which attach to headgear, but their conventional design is still susceptible to issues with stability and security. Additionally, some conventional chin straps have a top strap extending across the top of the head, but cannot be integrated with headgear and thus lack any functional connection.

Therefore, the need exists for a device having a chin strap which avoids the complete encircling of the head and is adjustable for customization by the patient. Furthermore, the need exists for a device having a chin strap which may be used in tandem with existing headgear of facemasks and thereby becoming an integrated part of the headgear. More particularly, there exists the need for a device which utilizes a top strap of conventional headgear such that the device and the top strap function as a unitary, continuous member. Such device may be compatible with most or all existing styles and brands of headgear and corresponding full-face masks, while also taking advantage of the securing properties inherent in the headgear.

### BRIEF SUMMARY OF THE INVENTION

The present invention generally comprises an apparatus, referred to generally hereafter as a mandibular positioning device, adapted for use with headgear for CPAP therapy face masks. In particular, a mandibular positioning device may be integrated with existing headgear to provide support to a user's chin and maintain the position of the user's mandible during sleep. While the user is asleep (and undergoing CPAP therapy), the device may prevent the user's mandible from shifting in a posterior direction (i.e., toward the back of the user's head), thus preventing displacement of the mask around the user's nose and/or mouth. It is to be appreciated, and in contrast with conventional devices, a mandibular positioning device is configured to be integrated with headgear, utilizing stabilizing properties inherent in the headgear and functioning as a unitary device rather than independent devices.

As used herein, the terms “anterior”, “posterior”, “proximal”, and “distal”, or other like terms, are provided to describe the position of a mandibular positioning device or headgear (referred to hereinafter as a “headgear device”), as well as components thereof, relative to a user's head, when a device is being worn by the user. These terms are intended to provide a frame of reference and are not intended to limit the scope of the present invention.

According to some embodiments of the present invention, a mandibular positioning device may include a first saddle, a second saddle, and a support strap, wherein the first saddle and the second saddle may each be configured to be attached to a portion of a headgear device for securing a face mask to a user's face. In some embodiments, a top edge of each saddle may be generally straight along the entire length of each saddle, whereas a bottom edge of each saddle may be straight along a first portion of the length of each saddle and curved in a downward direction (i.e., away from the top edge) along a second portion of the length of each saddle. Thus, a vertical width of each saddle (i.e., the distance between the top edge and the bottom edge) may increase from an anterior end to a posterior end, generally forming an “L” or “J” shape (depending on the frame of reference), with each saddle being a mirror image of the other.

In some embodiments, the first saddle and the second saddle may each comprise a proximal portion and a distal portion which may be joined together (removably or fixedly) along the length of the saddles, near the top edge and the bottom edge thereof, thereby forming an opening therebetween. When the device is attached to (or otherwise integrated with) a headgear device, a first lateral arm and a second lateral arm of the headgear device (i.e., the lateral arms which may be positioned near the temples of a user's head when worn) may be disposed within the opening of the first saddle and the opening of the second saddle, respectively.

According to some embodiments, the first saddle and the second saddle may each comprise one or more headgear fasteners on an interior of the proximal and distal portions, near the top edge thereof, for securing the mandibular positioning device to the headgear device. In some aspects, a headgear fastener may comprise a male fastener on the interior of the distal portion and a female fastener on the interior of the proximal portion (or vice versa), wherein the male and female fasteners may be joined together to attach the distal portion to the proximal portion.

In some embodiments of the present invention, the first saddle and the second saddle may each include one or more support strap fasteners for attaching the support strap thereto. More particularly, and in accordance with some embodiments, the first saddle may have a support strap fastener on an exterior of the distal portion to which a first end of the support strap may be attached. Likewise, the second saddle may have a support strap fastener on an exterior of the distal portion to which a second end of the support strap may be attached. In some embodiments, a support strap fastener may comprise a male (female) fastener configured to be attached to a female (male) fastener. According to some aspects, the support strap fastener may be disposed near the top edge of the first (second) saddle and toward an anterior end thereof.

According to some embodiments of the present invention, the support strap may be formed as a thin, flexible band generally comprising a “U” shape configured to support a user's lower jaw with a medial portion configured to be disposed underneath a user's chin when being worn by the user. In some embodiments, the support strap may have a plurality of removable partitions at each of the first end and the second end of the support strap for adjusting the length of the support strap. A first partition may be separated from an adjacent second partition by cutting, tearing, or otherwise separating along a partition line between the first and second partitions, thereby shortening the overall length of the support strap. When a user is wearing the mandibular positioning device, the support strap may be stabilized by a force applied by the support strap on the user's chin, generally in an upward direction, and by the engagement of each of the first saddle and the second saddle with the headgear device, which may stabilize the support strap in the anterior and posterior directions.

According to some embodiments, at each of the first end and the second end of the support strap, and, in some embodiments, on an interior portion thereof, may be one or more fasteners for engaging one or more strap fasteners of the first saddle and the second saddle, respectively. In some aspects, a fastener of the support strap may comprise a male (female) fastener configured to be attached to the female (male) fastener on the exterior of the distal portion of the saddles. In other embodiments, the support strap may be unitarily formed with each of the first and second saddles.

According to some implementations of the present invention, the mandibular positioning device may be attached to a headgear device by means of the first and second saddles. To attach the first and second saddles, a user may first separate, near the top edge thereof, the proximal and distal portions of each saddle (e.g., by disengaging the male (female) fasteners from the female (male) fasteners on the interior of the proximal and distal portions, respectively) to expose the openings between the proximal and distal portions. Following, the user may position a first and a second lateral arm of the headgear device which reside on each side of the user's head within the exposed openings of the first and second saddles, respectively. Once positioned, the proximal and distal portions may be rejoined along near the top edge of each saddle (e.g., by engaging the male (female) fasteners with the female (male) fasteners on the interior of the proximal and distal portions, respectively), thereby securing the headgear device within the openings of each saddle. The user may then insert one or both ends of the support strap underneath the first and/or second lateral arm of the headgear device, with the support strap toward the front of the user's lower jaw. Optionally, the user may first attached the support strap to a saddle before inserting an end of the support strap underneath a lateral arm of the headgear device. Following, the user may attach the first and second ends of the support strap to the first and second saddles, respectively (e.g., by engaging the male (female) fasteners of the support strap with the female (male) fasteners on the exterior of the first and second saddles), where the points of attachment of the support strap to the first and second saddles may be determined by the user's desired fit. Optionally, and prior to the attachment of the support strap, the user may remove one or more partitions from the first and/or second end of the support strap to shorten the support strap prior to attachment.

According to some embodiments of the present invention, an apparatus for stabilizing a mandibular position during CPAP therapy may comprise a) a first saddle and a second saddle, each of which may comprise i) a proximal portion having a first headgear fastener and ii) a distal portion having a second headgear fastener and a support strap fastener, and b) a support strap which may comprise i) a first end having a fastener which may be configured to engage the support strap fastener of the first saddle and ii) a second end having a fastener which may be configured to engage the support strap fastener of the second saddle, wherein the first saddle may be configured to receive a first lateral arm of a headgear device within an opening disposed between the proximal portion and the distal portion of the first saddle, wherein the second saddle may be configured to receive a second lateral arm of the headgear device within an opening disposed between the proximal portion and the distal portion of the second saddle, wherein the first headgear fastener and the second headgear fastener of the first saddle may be configured to retain the first lateral arm of the headgear device within the opening, and wherein the first headgear fastener and the second headgear fastener of the second saddle may be configured to retain the second lateral arm of the headgear device within the opening. In some embodiments, a vertical width of the first saddle may increase from an anterior end to a posterior end. In some embodiments, a width of the support strap may increase from each of the first end and the second end toward a medial portion of the support strap. In some embodiments, the support strap may further comprise a plurality of removable partitions at the first end for adjusting a length of the support strap.

According to some embodiments of the present invention, a system for stabilizing a mandibular position during CPAP therapy may comprise a mandibular positioning apparatus and a headgear device having a first lateral arm and a second lateral arm, wherein the apparatus may comprise a) a first saddle and a second saddle, each of which may comprise i) a proximal portion, ii) a distal portion, iii) a pair of headgear fasteners, and iv) a support strap fastener, and b) a support strap which may comprise i) a first end removably engaged with the first saddle and ii) a second end removably engaged with the second saddle, wherein the first lateral arm of the headgear device may be retained within an opening between the proximal portion and the distal portion of the first saddle, and wherein the second lateral arm of the headgear device may be retained within an opening between the proximal portion and the distal portion of the second saddle. In some embodiments, the headgear device may further comprise a third lateral arm and a first portion of the support strap may be disposed underneath the third lateral arm. In some embodiments, the headgear device may further comprise a fourth lateral arm and a second portion of the support strap may be disposed underneath the third lateral arm. In some embodiments, the headgear device may further comprise a top arm extending between the first lateral arm and the second lateral arm. In some embodiments, near the first saddle, a longitudinal axis of the top arm may be aligned with a longitudinal axis of the support strap. In some embodiments, an elongated edge of the support strap may be about parallel to an elongated edge of the top arm of the headgear device. In some embodiments, the support strap fastener of each saddle may be aligned with the top arm of the headgear device.

According to some implementations of the present invention, a method for engaging a mandibular positioning apparatus, which may have a first saddle, a second saddle, and a support strap, to a headgear device, which may have a plurality of lateral arms, may comprise the steps of a) separating, along a top edge of each saddle, a proximal portion and a distal portion of each saddle to expose an opening therebetween, b) positioning a first of the lateral arms of the headgear device within the opening of the first saddle, c) positioning a second of the lateral arms of the headgear device within the opening of the second saddle, d) joining, along the top edge of each saddle, the proximal portion and the distal portion of each saddle, e) positioning a first portion of the support strap underneath a third of the lateral arms of the headgear device, f) positioning a second portion of the support strap underneath a fourth of the lateral arms of the headgear device, g) engaging a first end of the support strap to the first saddle, and h) engaging a second end of the support strap to the second saddle. In some implementations, before the step of engaging the first end of the support strap to the first saddle, a length of the support strap may be adjusted by removing at least one partition from an end of the support strap. In some implementations, the step of separating the proximal portion and the distal portion of each saddle may comprise disengaging a first headgear fastener on each proximal portion from a second headgear fastener on each distal portion. In some implementations, the step of joining the proximal portion and the distal portion of each saddle may comprise attaching the first headgear fastener to the second headgear fastener. In some implementations, the step of engaging the first end of the support strap to the first saddle may comprise attaching a fastener on the first end to a first support strap fastener on the proximal portion of the first saddle. In some implementations, the headgear device may further comprise a top arm extending between the first of the lateral arms and the second of the lateral arms and the step of positioning the first of the lateral arms of the headgear device within the opening of the first saddle may comprise aligning the first support strap fastener with the top arm. In some implementations, the step of engaging the second end of the support strap to the second saddle may comprise attaching a fastener on the second end to a second support strap fastener on the proximal portion of the second saddle. In some implementations, the headgear device may further comprise a top arm extending between the first of the lateral arms and the second of the lateral arms and the step of engaging the first end of the support strap to the first saddle may comprise aligning the support strap with the top arm. In some implementations, the step of engaging the first end of the support strap to the first saddle may comprise aligning a longitudinal axis of the support strap with a longitudinal axis of the top arm.

### BRIEF DESCRIPTION OF THE DRAWINGS

FIG. 1 is a right perspective view diagram illustrating an exemplary headgear device attached to an exemplary face mask and an exemplary mandibular positioning device engaged thereto, each of which are being worn on a head of a user, in accordance with some embodiments of the present invention.

FIG. 2 is a left perspective view diagram illustrating the user, the face mask, the headgear device, and the mandibular positioning device of FIG. 1, in accordance with some embodiments of the present invention.

FIG. 3 is a side view diagram illustrating an exemplary first saddle of the mandibular positioning device illustrated in FIG. 1, in accordance with some embodiments of the present invention.

FIG. 4 is a side view diagram illustrating an exemplary second saddle of the mandibular positioning device illustrated in FIG. 1, in accordance with some embodiments of the present invention.

FIG. 5 is a side view diagram illustrating an exemplary mandibular positioning device engaged with an exemplary headgear device, each of which are being worn on a head (partially illustrated) of a user, in accordance with some embodiments of the present invention.

### DETAILED DESCRIPTION OF THE INVENTION

The invention, in its various aspects, will be explained in greater detail below. While the invention will be described in conjunction with several exemplary embodiments, the exemplary embodiments themselves do not limit the scope of the invention. Similarly, the exemplary illustrations in the accompanying drawings, where like elements have like numerals, do not limit the scope of the exemplary embodiments and/or invention, including any length, angles, or other measurements provided. Rather the invention, as defined by the claims, may cover alternatives, modifications, and/or equivalents of the exemplary embodiments.

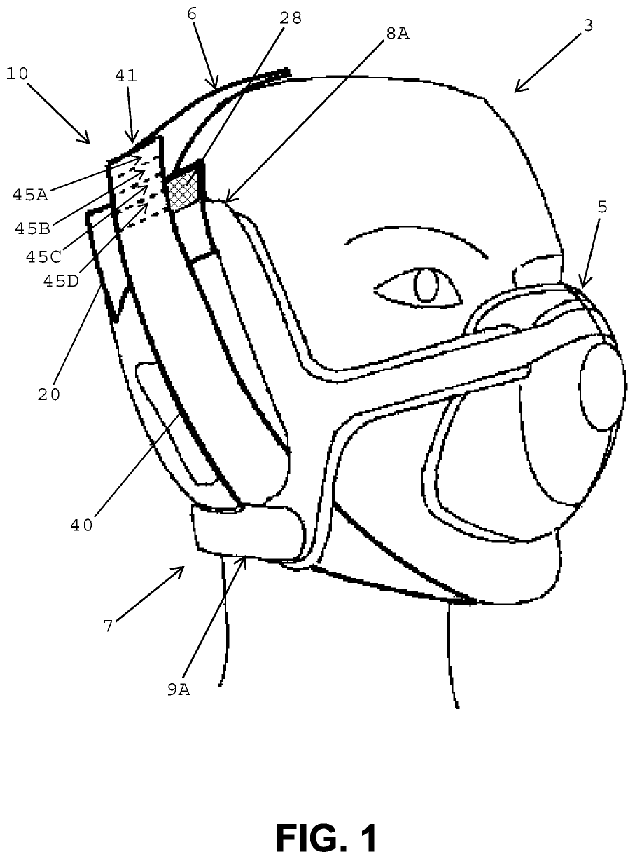

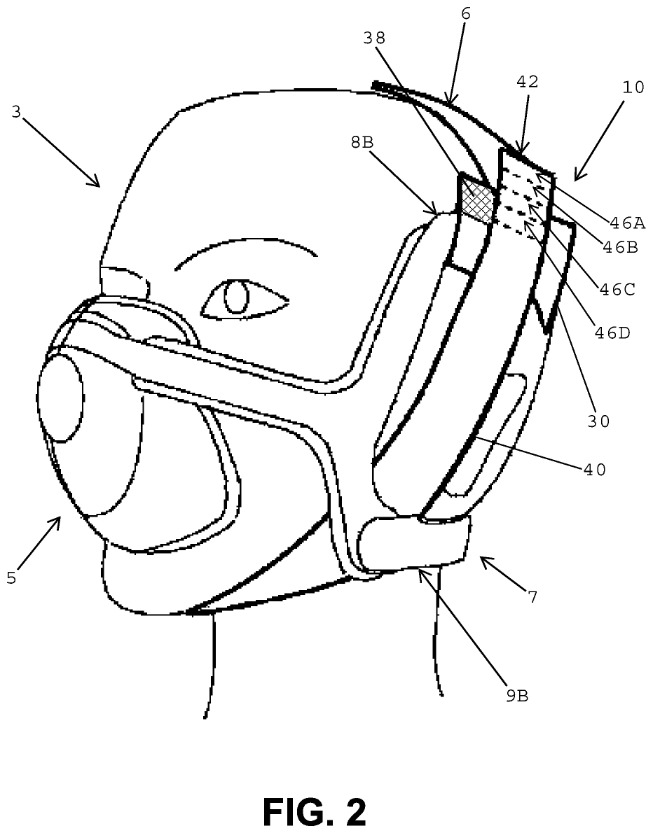

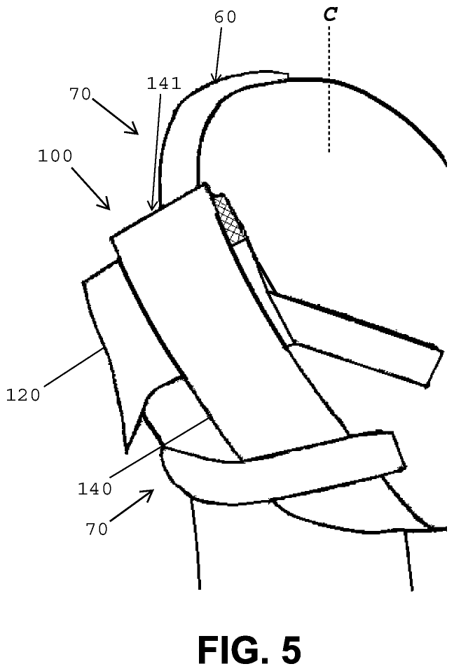

FIGS. 1 and 2 illustrate a user 3 wearing a face mask 5, which may be secured to the head of user 3 by a headgear device 7, and an exemplary mandibular positioning device 10 engaged with headgear device 7 at the sides of the head of user 3. According to some embodiments, a headgear device may have a plurality of straps which extend around a user's head and/or face. For example, as further illustrated in FIGS. 1 and 2, headgear device 7 may comprise upper lateral arms 8A and 8B, lower lateral arms 9A and 9B, and a top arm 6 extending between upper lateral arms 8A and 8B (another exemplary headgear device 70 is also illustrated in FIG. 5).

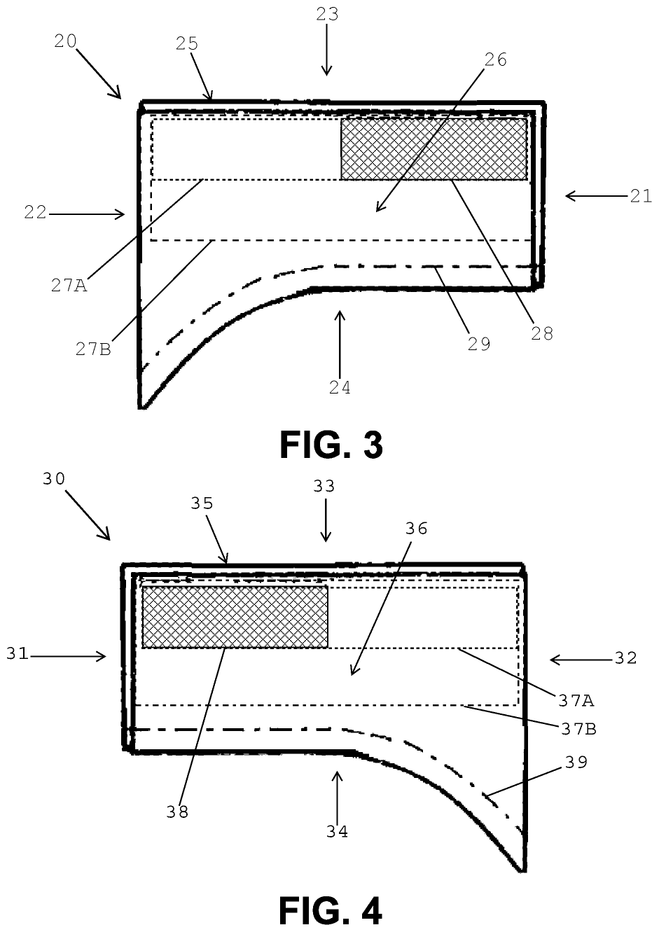

As further illustrated in FIGS. 1 and 2, and in accordance with some embodiments of the present invention, mandibular positioning device 10 may include a first saddle 20 (illustrated in FIG. 1), a second saddle 30 (illustrated in FIG. 2), and a support strap 40. In some embodiments, and as more clearly illustrated in FIGS. 3 and 4, a top edge 23 and 33 of first saddle 20 and second saddle 30, respectively, may be generally straight along the entire length thereof, whereas a bottom edge 24 and 34 of first saddle 20 and second saddle 30, respectively, may be straight along a first portion of the length thereof and curved in a downward direction (i.e., toward the neck of user 3, relative to the positions illustrated in FIGS. 1 and 2) along a second portion thereof. Thus, a vertical width of first saddle 20 and second saddle 30—that is, the distances between top edge 23 and bottom edge 24 and between top edge 33 and bottom edge 34, respectively—may increase from an anterior end 21 (right side of FIGS. 3) and 31 (left side of FIG. 4), respectively, to a posterior end 22 (left side of FIGS. 3) and 32 (right side of FIG. 4), respectively. Though, according to FIGS. 3 and 4, first saddle 20 and second saddle 30 are illustrated as mirror images of each other, it is to be appreciated that each saddle may differ in size and/or shape. Furthermore, and according to some embodiments, each saddle may be identical to the other such that they may be interchangeable. Additionally, it is to be appreciated that a saddle may be adapted to accommodate various headgear device designs. For example, the width of a saddle may increase in a posterior direction such that its top edge is upwardly curved and its bottom edge is downwardly curved (e.g., to accommodate headgear device straps which widen toward the back of a user's head). In another example, a saddle may comprise multiple openings (e.g., at an anterior and/or posterior end) which may accommodate headgear devices having more than one strap extending along the side of a user's head.

In some embodiments, and as further illustrated in FIGS. 3 and 4, first saddle 20 and second saddle 30 may each comprise a proximal portion 25 and 35, respectively (i.e., the portions closest to the head of user 3 in FIGS. 1 and 2), and a distal portion 26 and 36, respectively (i.e., the portions furthest from the head of user 3 in FIGS. 1 and 2), which may be joined together along the lengths of first saddle 20 and second saddle 30 near top edge 23 and 33, respectively, and bottom edge 24 and 34, respectively. According to some embodiments, and as illustrated in FIG. 3, proximal portion 25 (only the top of which is visible in FIG. 3) may be joined together with distal portion 26, near bottom edge 24, along a seam 29 (denoted by a dash-dotted line). Likewise, and as illustrated in FIG. 4, proximal portion 35 (only the top of which is visible in FIG. 4) may be joined together with distal portion 36, near bottom edge 34, along a seam 39 (denoted by a dash-dotted line). When proximal portion 25 and 35 is joined together with distal portion 26 and 36, respectively, an opening may be formed therebetween extending from anterior end 21 and 31 to posterior end 22 and 32, respectively. As illustrated in FIG. 1, for example, when mandibular positioning device 10 is engaged with headgear device 7, a portion of upper lateral arm 8A of headgear device 7 may be disposed within the opening of first saddle 20. Likewise, as illustrated in FIG. 2, for example, when mandibular positioning device 10 is engaged with headgear device 7, a portion of upper lateral arm 8B of headgear device 7 may be disposed within the opening of second saddle 30.

According to some embodiments, and as further illustrated, for example, in FIGS. 3 and 4, first saddle 20 and second saddle 30 may comprise headgear fasteners 27A and 27B and 37A and 37B, respectively, for attaching device 10 to headgear device 7. As more clearly illustrated in FIG. 3, for example, headgear fasteners 27A (denoted by a small dotted line depicting its position on the interior of first saddle 20) and 27B (denoted by a large dotted line depicting its position on the interior of first saddle 20) may be disposed on an interior of proximal portion 25 and on an interior of distal portion 26, respectively. Likewise, as more clearly illustrated in FIG. 4, for example, headgear fasteners 37A (denoted by a small dotted line depicting its position on the interior of second saddle 30) and 37B (denoted by a large dotted line depicting its position on the interior of second saddle 30) may be disposed on an interior of proximal portion 35 and on an interior of distal portion 36, respectively. In accordance with some embodiments, and as further illustrated in FIGS. 3 and 4, headgear fasteners 27A and 37A may be about the same length as, but narrower than, headgear fasteners 27B and 37B. This may allow the proximal portion and distal portion of each saddle to be attached at various positions. It is to be appreciated, however, that headgear fasteners 27A, 27B, 37A, and 37B may comprise various shapes and sizes which may be the same or different between two or more of the headgear fasteners.

In some embodiments, headgear fasteners 27A and 37A may comprise a male fastener and headgear fasteners 27B and 37B may comprise a female fastener (or vice versa), which can be joined together to attach proximal portion 25 and 35 to medial portion 26 and 36, respectively. For example, and without limitation, headgear fasteners 27A, 27B, 37A, and 37B may comprise “hook-and-loop” type fasteners in which headgear fasteners 27A and 37A may each comprise a plurality of hooks configured to mate with a plurality of loops of headgear fasteners 27B and 37B, respectively. It is to be appreciated, however, that other types of fasteners may be used, such as, and without limitation, snap type fasteners, zipper type fasteners, or button type fasteners.

As further illustrated in FIGS. 3 and 4, and in accordance some embodiments of the present invention, first saddle 20 and second saddle 30 may comprise a support strap fastener 28 and 38, respectively, for attaching support strap 40 thereto. Support strap fastener 28 may be disposed on an exterior of distal portion 26 to which a first end 41 of support strap 40 may be attached (illustrated, e.g., in FIG. 1). Likewise, second saddle 30 may have a support strap fastener 38 on an exterior of distal portion 36 to which a second end 42 of support strap 40 may be attached (illustrated, e.g., in FIG. 2). In some embodiments, and similar to headgear fasteners 27A, 27B, 37A, and 37B, support strap fastener 28 and 38 may comprise a male fastener configured to mate with a female fastener of support strap 40 (or vice versa). According to some aspects, and as further illustrated in FIGS. 3 and 4, support strap fastener 28 and 38 may be disposed near top edge 23 and 33 of first saddle 20 and second saddle 30 and toward anterior end 21 and 31 thereof, respectively.

Referring back, generally to FIGS. 1 and 2, according to some embodiments of the present invention, support strap 40 may be formed as a thin band generally comprising a “U” shape (when worn by user 3) which may support the lower jaw of user 3 and which may comprise a medial portion disposed underneath the chin of user 3. In some embodiments, the shape of a support strap may be configured according to the anatomical shape of a user's chin. For example, as further illustrated in FIGS. 1 and 2, the width of support strap 40 may increase from first end 41 and second end 42 towards the medial portion. It is to be appreciated, however, according to other embodiments, a support strap may have a fixed width along the entirety thereof, or may be narrower or wider near a medial portion (e.g., to accommodate different chin sizes). It is also to be appreciated that, when user 3 is wearing mandibular positioning device 10, support strap 40 may provide a force generally in an upward direction (i.e., toward the head of user 3), which may help stabilize the chin of user 3 in a vertical direction. Additionally, support strap 40 may be further stabilized in an anterior and posterior direction by the engagement of mandibular positioning device 10 with headgear device 7 (via first saddle 20 and second saddle 30), which provides a relatively stable point of attachment (as compared to, for example, engaging mandibular positioning device 10 around the top of the head of user 3).

As further illustrated in FIGS. 1 and 2, and according to some embodiments, support strap 40 may have a plurality of removable partitions 45A-D (illustrated in FIGS. 1) and 46A-D (illustrated in FIG. 2), separated by partition lines (denoted by broken lines), at first end 41 and second end 42, respectively, which may allow user 3 to adjust the length of support strap 40. For example, and without limitation, partition 45A may be separated from support strap 40 by cutting, tearing, or otherwise separating along the partition line between partition 45A and partition 45B, thereby shortening the overall length of support strap 40.

According to some embodiments of the present invention, near each of first end 41 and second end 42 of support strap 40, and along a portion of an interior of support strap 40 (not illustrated), may be one or more fasteners (not illustrated) for engaging support strap fastener 28 and 38 of first saddle 20 and second saddle 30, respectively. As described above, fasteners near first end 41 and second end 42 may comprise a male (female) fastener configured to mate with a female (male) support strap fastener 28 and 38, respectively. It is to be appreciated, however, that according to other embodiments, a support strap may be unitarily formed with each of the first and second saddles, or may be fixedly attached thereto.

According to some implementations of the present invention, and with reference, generally to FIGS. 1-4, mandibular positioning device 10 may be attached to headgear device 7 by means of first saddle 20 and second saddle 30 (as illustrated, for example, in FIGS. 1 and 2). To engage first saddle 20 and second saddle 30 with headgear device 7, user 3 may first separate proximal portion 25 and 35 from distal portion 26 and 36, respectively, by disengaging headgear fastener 27A and 37A from headgear fastener 27B and 37B, respectively. By disengaging the headgear fasteners, the openings between proximal portion 25 and 35 and distal portion 26 and 36, respectively, may be exposed to receive portions of upper lateral arms 8A and 8B, respectively, of headgear device 7 which reside on the side of the head of user 3 when worn. Following, user 3 may position upper lateral arms 8A and 8B within the exposed openings of first saddle 20 and second saddle 30, respectively, with each saddle being oriented such that top edge 23 and 33 are closest to the head of user 3, relative to bottom edge 24 and 34. Once positioned, proximal portion 25 and 35 may be rejoined with distal portion 26 and 36, respectively, by engaging headgear fastener 27A and 37A with headgear fastener 27B and 37B, respectively, thus securing upper lateral arms 8A and 8B of headgear device 7 within the openings of first saddle 20 and second saddle 30.

In preferred implementations, first saddle 20 and second saddle 30 may be positioned such that support strap fasteners 28 and 38, respectively, are aligned with top arm 6 of headgear 7 extending between upper lateral arms 8A and 8B. According to some implementations, prior to engaging first saddle 20 and second saddle 30 with headgear device 7, a user may first wear headgear device 7 with face mask 5 and adjust headgear device 7 until it is secure on the head of user 3. In some aspects, user 3 may run air through face mask 5 (e.g., by turning on a CPAP machine connected therewith) to determine if any air can escape around face mask 5 and, if needed, user 3 may further adjust headgear device 7. Once secure, user 3 may remove headgear device 7 and face mask 5 and then proceed to engage first saddle 20 and second saddle 30 with headgear device 7.

According to some implementations, following the engagement of first saddle 20 and second saddle 30 with headgear device 7, user 3 may place headgear device 7 (now equipped with first saddle 20 and second saddle 30) and face mask 5 back around the head and face of user 3. Next, user 3 may insert first end 41 and second end 42 underneath lower lateral arm 9A and lower lateral arm 9B, respectively, positioning the medial portion of support strap 40 toward a front portion of the lower jaw of user 3 and bringing first end 41 to first saddle 20 and second end 42 to second saddle 30 such that portions of support strap 40 are disposed between the lower jaw of user 3 and each of lower lateral arms 9A and 9B of headgear device 7 (optionally, a user may instead attach the support strap to one of the saddles before inserting an end underneath a lower lateral arm). Following, user 3 may attach first end 41 and second end 42 of support strap 40 to first saddle 20 and second saddle 30, respectively, by engaging the fastener near first end 41 with support strap fastener 28 of first saddle 20 and by engaging the fastener near second end 42 with support strap fastener 38 of second saddle 30. In some implementations, user 3 may pull up on each of first end 41 and second end 42 and/or open their mouth (i.e., to lower the chin) to create tension in support strap 40 prior to attachment to first saddle 20 and second saddle 30.

In preferred implementations, support strap 40 may be positioned such that it is generally aligned with top arm 6, with the elongated edges of support strap 40 (i.e. the edges extending along the sides and lower jaw of the head of user 3) and the elongated edges of top arm 6 (i.e., the edges extending across the top of the head of user 3) being about parallel near first saddle 20 and second saddle 30. For example, when viewed from a side of a user's head and near a saddle, a central longitudinal axis of a top strap may be generally aligned with a central longitudinal axis of a support strap. Therefore, depending on a type of headgear device, the ends of a support strap may positioned relatively closer to a back, center, or front of a user's head. For example, and as illustrated in FIG. 5, a headgear device 70 may have a top arm 60 which extends around a head and which has a medial portion disposed posteriorly from a center C of the head. In this case, an end 141 of a support strap 140 may be attached to a saddle 120 at a more posterior position relative to an end of a support strap which may be engaged (via a saddle) to a headgear device having a top arm which extends across a center of a head.

In accordance with some implementations, and with further reference to FIGS. 1-4, the points of attachment of support strap 40 to first saddle 20 and second saddle 30 may be determined by the desired fit of user 3 and thus the attachment points may be relatively closer to, or further from, a top edge of each of first end 41 and second end 42. Optionally, and prior to the attachment of support strap 40, user 3 may remove one or more of partitions 45A-D and/or 46A-D near first end 41 and/or second end 42 (e.g., by cutting along a partition line) in order to shorten support strap 40 prior to attachment.

It is to be understood that variations, modifications, and permutations of embodiments of the present invention may be made without departing from the scope thereof. It is also to be understood that the present invention is not limited by the specific embodiments, descriptions, or illustrations or combinations of either components or steps disclosed herein. Thus, although reference has been made to the accompanying figures, it is to be appreciated that these figures are exemplary and are not meant to limit the scope of the invention. In particular, it is to be appreciated that a mandibular positioning device may be integrated with, or may otherwise be adapted to be used with, a variety of headgear devices and should not be limited to the examples provided herein.

What is claimed is:

1. An apparatus for stabilizing a mandibular position during CPAP therapy, comprising:
a) a first saddle and a second saddle, each comprising i) a proximal portion having a first headgear fastener and ii) a distal portion having a second headgear fastener and a support strap fastener; and
b) a support strap comprising i) a first end having a fastener configured to engage said support strap fastener of said first saddle and ii) a second end having a fastener configured to engage said support strap fastener of said second saddle;
wherein said first saddle is configured to receive a first lateral arm of a headgear device within an opening disposed between said proximal portion and said distal portion of said first saddle,
wherein said second saddle is configured to receive a second lateral arm of said headgear device within an opening disposed between said proximal portion and said distal portion of said second saddle,
wherein said first headgear fastener and said second headgear fastener of said first saddle are configured to retain said first lateral arm of said headgear device within said opening, and
wherein said first headgear fastener and said second headgear fastener of said second saddle are configured to retain said second lateral arm of said headgear device within said opening.

2. The apparatus of claim 1, wherein a vertical width of said first saddle increases from an anterior end to a posterior end.

3. The apparatus of claim 1, wherein a width of said support strap increases from each of said first end and said second end toward a medial portion of said support strap.

4. The apparatus of claim 1, wherein said support strap further comprises a plurality of removable partitions at said first end for adjusting a length of said support strap.

5. A system for stabilizing a mandibular position during CPAP therapy, comprising a mandibular positioning apparatus and a headgear device having a first lateral arm and a second lateral arm, said apparatus comprising:
a) a first saddle and a second saddle, each comprising i) a proximal portion, ii) a distal portion, iii) a pair of headgear fasteners, and iv) a support strap fastener; and
b) a support strap comprising i) a first end removably engaged with said first saddle and ii) a second end removably engaged with said second saddle;
wherein said first lateral arm of said headgear device is retained within an opening between said proximal portion and said distal portion of said first saddle, and
wherein said second lateral arm of said headgear device is retained within an opening between said proximal portion and said distal portion of said second saddle.

6. The system of claim 5, wherein said headgear device further comprises a third lateral arm and wherein a first portion of said support strap is disposed underneath said third lateral arm.

7. The system of claim 6, wherein said headgear device further comprises a fourth lateral arm and wherein a second portion of said support strap is disposed underneath said third lateral arm.

8. The system of claim 5, wherein said headgear device further comprises a top arm extending between said first lateral arm and said second lateral arm.

9. The system of claim 8, wherein, near said first saddle, a longitudinal axis of said top arm is aligned with a longitudinal axis of said support strap.

10. The system of claim 8, wherein an elongated edge of said support strap is about parallel to an elongated edge of said top arm of said headgear device.

11. The system of claim 8, wherein said support strap fastener of each saddle is aligned with said top arm of said headgear device.

12. A method for engaging a mandibular positioning apparatus to a headgear device,
said device having a plurality of lateral arms and said apparatus comprising a first saddle, a second saddle, and a support strap,
said method comprising the steps of:
a) separating, along a top edge of each saddle, a proximal portion and a distal portion of each saddle to expose an opening therebetween;
b) positioning a first of said lateral arms of said headgear device within said opening of said first saddle;
c) positioning a second of said lateral arms of said headgear device within said opening of said second saddle;
d) joining, along said top edge of each saddle, said proximal portion and said distal portion of each saddle;
e) positioning a first portion of said support strap underneath a third of said lateral arms of said headgear device;
f) positioning a second portion of said support strap underneath a fourth of said lateral arms of said headgear device;
g) engaging a first end of said support strap to said first saddle; and
h) engaging a second end of said support strap to said second saddle.

13. The method of claim 12, wherein, before the step of engaging said first end of said support strap to said first saddle, a length of said support strap is adjusted by removing at least one partition from an end of said support strap.

14. The method of claim 12, wherein said step of separating said proximal portion and said distal portion of each saddle comprises disengaging a first headgear fastener on each proximal portion from a second headgear fastener on each distal portion.

15. The method of claim 14, wherein said step of joining said proximal portion and said distal portion of each saddle comprises attaching said first headgear fastener to said second headgear fastener.

16. The method of claim 12, wherein said step of engaging said first end of said support strap to said first saddle comprises attaching a fastener on said first end to a first support strap fastener on said proximal portion of said first saddle.

17. The method of claim 16, wherein said headgear device further comprises a top arm extending between said first of said lateral arms and said second of said lateral arms and wherein said step of positioning said first of said lateral arms of said headgear device within said opening of said first saddle comprises aligning said first support strap fastener with said top arm.

18. The method of claim 12 wherein said step of engaging said second end of said support strap to said second saddle comprises attaching a fastener on said second end to a second support strap fastener on said proximal portion of said second saddle.

19. The method of claim 18, wherein said headgear device further comprises a top arm extending between said first of said lateral arms and said second of said lateral arms and wherein said step of engaging said first end of said support strap to said first saddle comprises aligning said support strap with said top arm.

20. The method of claim 19, wherein said step of engaging said first end of said support strap to said first saddle comprises aligning a longitudinal axis of said support strap with a longitudinal axis of said top arm.
